(19) United States
(12) Patent Application Publication
(10) Pub. No.: US 2018/0280108 A1
(43) Pub. Date: Oct. 4, 2018

(54) BREAST SUPPORT COMPRESSION PILLOW

(71) Applicant: Devicor Medical Products, Inc., Cincinnati, OH (US)

(72) Inventor: Bruce John Kevin, Burlington, KY (US)

(21) Appl. No.: 15/764,340

(22) PCT Filed: Sep. 29, 2016
(86) PCT No.: PCT/US16/54460

§ 371 Date: Mar. 29, 2018

Related U.S. Application Data
(60) Provisional application No. 62/234,883, filed on Sep. 30, 2015.

(51) Int. Cl. A61B 90/17; A61B 10/02; A61B 90/11

(52) U.S. Cl. CPC A61B 90/17; A61B 2017/00557; A61B 90/11; A61B 10/0233

(57) ABSTRACT

The invention provides an adjustable means for supporting the breast from beneath the nipple during a prone breast biopsy procedure. By increasing or decreasing the volume of air or fluid within the pillow, the clinician can apply a controlled amount of compression on the breast, thereby increasing or decreasing the compressed area of the breast. The invention is believed to have utility for all modalities of breast biopsy; however, it is believed to be especially useful for a prone MRI breast biopsy procedure.

### FIELD OF THE INVENTION

This invention is in the field of medical device equipment for conducting biopsy procedures. Specifically, it is in the field of breast support techniques for a patient undergoing a breast biopsy procedure.

### BACKGROUND OF THE INVENTION

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

The state of the art for breast biopsy is vacuum-assisted breast biopsy. A current textbook in this area is “Vacuum-Assisted Breast Biopsy with Mammotome®,” available Nov. 11, 2012, copyright 2013 by Devicor Medical Germany GmBh, published in Germany by Springer Medizin Verlag, Authors: Markus Hahn, Anne Tardivon and Jan Casselman, ISBN 978-3-642-34270-7.

Biopsy devices may be used under ultrasound image guidance, stereotactic (X-ray) guidance, MRI guidance, Positron Emission Mammography (“PEM” guidance), Breast-Specific Gamma Imaging (“BSGI”) guidance, or otherwise. Each procedure has its own methodology based on the form of imaging guidance used. The following briefly describes ultrasound image guided biopsy procedures, stereotactic guided biopsy procedures and MRI guided biopsy procedures.

In an ultrasound image guided breast biopsy procedure, the operator may position an ultrasound transducer on the patient's breast and maneuver the transducer while viewing an ultrasound image display screen to locate suspicious tissue in the patient's breast. Once the operator locates the suspicious tissue, the operator may anesthetize the target region of the breast. Once the breast has been anesthetized, the operator may create an initial incision using a scalpel at a location on the exterior of the breast offset from the transducer. A needle of a breast biopsy probe disposed coaxially within an introducer cannula is then inserted into the breast through the initial incision. The operator continues to hold the ultrasound transducer with one hand while maneuvering the biopsy probe with the other hand. While viewing the ultrasound image on the display screen, the operator guides the needle to a position adjacent to the suspicious tissue. A cutter within the needle of the probe is used to remove tissue which is then conveyed either to a manual pick-up location on the breast biopsy device or to a tissue sample chamber. The needle of the breast biopsy device is then removed, leaving the introducer cannula disposed within the breast. The introducer cannula may then be used to introduce a biopsy marker cannula for deploying a biopsy site marker at the biopsy site. Once a marker has been deployed at the biopsy site, the biopsy marker cannula and the introducer cannula are both removed from the breast and the incision is closed using a medically acceptable way to close breaks in the skin.

In a stereotactic image guided breast biopsy procedure, the patient is first positioned relative to x-ray equipment, which includes a breast localization assembly. In some procedures, the patient is oriented in a prone position, with the patient lying face down on a procedure table with at least one breast hanging pendulously through an aperture in the procedure table. The breast is then compressed between a compression paddle and an x-ray receptor of a localization assembly that is positioned under the procedure table. A breast biopsy device is positioned on an automatic guide device in front of the compression paddle and between the breast and an x-ray source. Once positioning of the patient and localization of the breast are complete, a scout image is acquired with the x-ray receptor in a zero-degree angular position (i.e., the x-rays are emitted along an axis normal relative to the x-ray receptor). If the scout image indicates that the patient has been positioned in a desired position, the procedure may proceed with the acquisition of stereotactic image pairs. Stereotactic image pairs are acquired by orienting the x-ray source at various complementary angular positions relative to the x-ray receptor (e.g., +15° and −15°), with at least one x-ray image acquired at each position.

Further in the stereotactic image guided breast biopsy procedure, once a suitable stereotactic image pair is acquired, an operator may identify a target site where biopsy sampling is desired by examining the stereotactic image pair. The target site is marked on each stereotactic image and a precise location of the target site on a Cartesian coordinate system is computed using an image processing module. The computed location of the target site is then communicated to the automatic guide device. The automatic guide device is responsive to this information to position the breast biopsy probe into a position that aligns with the target site. With the breast biopsy device positioned, an operator may then fire a needle of the biopsy probe into the breast of the patient, thereby positioning the needle at the target site. A cutter within the needle of the probe is used to remove tissue, which is then conveyed either to a manual pick-up location on the breast biopsy device or to a tissue sample chamber. After the biopsy tissue is removed, a biopsy marker cannula is inserted into the needle and is used to deploy a biopsy site marker at the biopsy site. Once a marker has been deployed at the biopsy site, the needle is removed from the breast and the incision is closed using a medically acceptable way to close breaks in the skin.

In an MRI guided breast biopsy procedure, after the patient is properly positioned on the table and a targeting device (e.g., a grid and cube combination or a pillar, post and cradle support combination) has been deployed and used, a baseline MRI image is taken to verify the target location. After that, a scalpel is used to incise the skin of the breast. Next, an assembly, formed by an obturator disposed in a sleeve, is inserted through the incision to penetrate the breast tissue under the skin. In some acceptable surgical techniques, the obturator is removed and an imaging rod is inserted into the sleeve in place of the obturator. An imaging rod is defined simply as an appropriately shaped rod that includes a feature that is detectable by an imaging technique being used for the biopsy procedure. The MRI image of the imaging rod is used to locate the site to which the sleeve/obturator assembly has penetrated. In some other acceptable surgical techniques, the obturator cooperates with the breast tissue to provide a visually observable artifact in an MRI image. With both of these techniques, after the location within the breast where the biopsy is to be taken is confirmed, the obturator or the imaging rod is removed.

Further in the MRI guided breast biopsy procedure, after the obturator or imaging rod has been removed, it is replaced in the sleeve with the needle of a breast biopsy probe. A cutter within the needle of the probe is used to remove tissue, which is then conveyed either to a manual pick up location on the breast biopsy device or to a breast biopsy device sample chamber. After the biopsy tissue is removed, a biopsy marker cannula is inserted into the needle and is used to deploy a biopsy site marker at the biopsy site. The needle is then removed from the sleeve. Optionally, the imaging rod or the obturator is put back into the breast for reimaging of the biopsy site. Then the imaging rod or obturator and the sleeve are removed.

Published US Patent Application 2013/0116570, “Local Compression During Automated Ultrasound Scanning and Methods of Acoustic Coupling”, published on 9 May 2013 describes and claims a system for imaging a portion of a body, such as a human breast. The system comprises a first scanning system that ultrasonically scans a portion of a body while a compression system applies a compressing force to the portion of the body being scanned. A device is used for containing an acoustic coupling gel for enhanced acoustic coupling with the portion of body.

U.S. Pat. No. 5,499,989, entitled “Breast Biopsy Apparatus and Method of Use”, issued on 19 Mar. 1996; it describes and claims an apparatus and method for obtaining samples of suspicious breast tissue. The breast is placed between two plates, which compress, and therefore stabilize the tissue. The upper compression plate has an aperture therein that allows for the placement of a guide spool having a flesh adhering surface thereon onto the compressed breast itself. Markings on the spool allow for accurate placement using the cross hairs, or laser light pointer, of the mammographic unit. For additional placement verification, the guide spool is radiopaque and, thus, an X-ray taken directly down through the aperture will aid in ascertaining if the spool is properly placed. A tubular punch is advanced and rotated, cutting through the tissue, which is recovered in the tube. Alternatively, a localizing needle can be placed with its tip proximate the calcification, a guide wire mandrel having the same diameter as the guide spool aperture placed over it, the guide spool placed and aligned, the mandrel removed, and the tubular punch is inserted and advanced as above, removing the tissue in question, along with the localizing needle. The disclosure of this U.S. patent is incorporated by reference herein.

U.S. Pat. No. 5,971,998, entitled “Support Device and Method for Controlling Breast Thickness During Stereotactic Guided Needle Biopsy”, issued on 26 Oct. 1999, describes and claims a support device for controlling breast thickness during stereotactic guided needle biopsy. The support device includes a compressible support having an inner surface which extends adjacent to a substantial portion of a peripheral surface of a patient's breast when the breast and the support are disposed between the fixed and pressure plates of an apparatus for performing stereotactic guided needle biopsy. The compressible support restricts the inferior and lateral excursion of the breast as the plates are moved toward one another and the patient's breast and the compressible support are pressed between the plates. A method for controlling breast thickness during stereotactic guided needle biopsy is also provided. The disclosure of this U.S. patent is incorporated by reference herein.

US Published Patent Application US2005/0265518 “Mammography procedure and apparatus for reducing pain when compressing a breast” published on 1 Dec. 2005. It describes and claims a method and apparatus for compressing a patient's breast when using an X-ray mammography machine to take an image wherein said machine has a compression paddle and a bucky. A movable interface plate is mounted on the bucky as an interface between the bucky and a patient's breast. The method includes a step wherein the compression paddle is moved downwardly to provide compression forces on the breast; the movement of the compression paddle is stopped at a position where less than the full desired compression of the breast is attained. Next, the movable interface plate is elevated upwardly against the breast to obtain the full desired compression. The upward movement of the interface plate functions to distribute and balance the compression and shear forces applied to the breast.

U.S. Pat. No. 7,489,761, entitled “Breast compression for digital mammography, tomosynthesis and other modalities” issued on 10 Feb. 2009, describes and claims a breast x-ray imaging method and system that is particularly suited for tomosynthesis imaging but also is useful for conventional mammography. A fluid containing pillow or bag is placed between the breast and a paddle that compresses the breast against a breast platform covering an imaging device, to enhance patient comfort and provide other benefits. Alternatives include a flexible sheet compressing the breast, and a compressible foam, preferably contoured to accommodate a patient's breast. The disclosure of this U.S. patent is incorporated by reference herein.

US Published Patent Application 20120143083, entitled “Devices and Methods for Improving the Usability Of Stereotactic Imaging For Performing a Breast Biopsy” published on 7 Jun. 2012. It describes and claims a device to distance a breast of a patient between a breast support plate and a compression plate while undergoing a stereotactic biopsy comprises a platform distanced from the breast support plate by a distancing structure connected with the platform when the device is mounted on the breast support plate. The device can be bound to the breast support plate to resist movement of the platform relative to the breast support plate. The platform is formed of a material that permits x-rays to pass through the platform substantially unobstructed such that no artifacts are introduced by the platform during imaging.

U.S. Pat. No. 8,241,302, entitled “Biopsy targeting cube with angled interface”, issued 14 Aug. 2013, describes and claims a biopsy system comprises a control module, a localization assembly, a biopsy device, and a targeting cube. The biopsy device comprises a probe and other components, which selectively couple with a targeting cube that is configured to selectively couple with a grid plate having apertures for receiving the targeting cube. The targeting cube comprises a body defined by faces. The targeting cube further comprises guide holes that originate and terminate at the faces and pass through the body of the targeting cube to provide passageways through the targeting cube. The faces of the targeting cube comprise a tapered profile from a proximal end to a distal end. The tapered profile of the targeting cube may be created by the faces themselves or by protruding elements from the faces. The body of the targeting cube and/or the protruding elements may be at least partially comprised of an elastomeric material. The disclosure of this U.S. patent is incorporated by reference herein.

The thesis entitled “Tissue Stabilization in MRI-Guided Breast Biopsy” was written by Behzad Iranpanah as his Master's Degree Thesis at McMaster University in Hamilton Ontario, Canada and published in August of 2013. The abstract for this thesis states: Breast cancer is the most common form of cancer in women in the United States. Histopathological examination through breast biopsy is considered as the “Gold Standard” for definitive diagnosis. Contrast-enhanced Magnetic Resonance Imaging (MRI) is often used for guiding the biopsy in those cases in which the tumor may not be detectable under Ultrasound or X-ray mammography. Stabilization of the breast tissue during the biopsy is critical for its success to ensure that the target would not be displaced due to the patient movement or tissue deformation. Conventionally, the breast tissue is immobilized by firmly compressing it between two parallel plates. However, high compression forces causes significant patient discomfort and can reduce the intake of the contrast agent, which negatively impact the image quality.

This thesis introduces devices and control methodologies for active tissue stabilization in magnetic resonance imaging (MRI)-guided breast biopsy. Pneumatic and piezoelectric actuators have been considered for developing concept designs for MRI-compatible tissue stabilization devices. Only the pneumatic device has been prototyped and tested. The device is comprised of two pneumatically-actuated support plates that would stabilize the biopsy target movements during needle insertion. An optimized geometry for the support plates allows for a good degree of tissue stabilization without relying on large compression forces. The plate configuration can also be adjusted inside the magnet bore using pneumatic actuators driven by pressure controlled valves that are placed in the MR control room. This capability allows for the compensation of the target displacement based on MR image feedback. When combined with a separate needle drive mechanism, this stabilization device would enable in-bore MR-guided breast biopsy in combination with an in-bore needle driver system. The proposed approach offers improved target stabilization at reduced compression force and patient discomfort that may also enhance MR image quality as result of greater intake of contrast agent. The open-front design of the stabilization plates provides greater flexibility in selecting the needle insertion entry point, and active adjustment of the support plates based on MR feedback improves the targeting accuracy.

U.S. Pat. No. 8,886,284, entitled “Devices and methods for combined optical and magnetic resonance Imaging” issued on 11 Nov. 2014, and describes and claims a system for combined optical imaging and magnetic resonance imaging of breast tissue, comprising: a Magnetic Resonance Imaging (MRI) system; a plurality of protrusions, formed on a housing, the housing adapted to be releasably secured to a grid such that each protrusion projects through a hole of the grid; each protrusion including an optical window covered by a material transparent to a preselected range of electromagnetic energy; the grid being a grid of a Magnetic Resonance Imaging (MRI) breast tissue compression system, wherein each protrusion is configured to releasably mates with a grid hole of the grid; a plurality of light sources coupled to at least some windows of the protrusions; a plurality of light detectors coupled to receive light from at least some windows of the protrusions; and a computer; wherein the computer is configured with firmware comprising machine readable instructions that, when executed, reconstructs a near-infrared (NIR) tomographic image of breast tissue from light received by the light detectors; wherein the firmware that reconstructs the NIR image is constrained by MRI breast image data acquired by the MRI imaging system; and wherein the magnetic resonance breast image data provides quantification data of one or more of lipid concentration, and water concentration, wherein the quantification data is used in reconstructing the NIR image, and wherein the NIR image further comprises quantification of deoxyhemoglobin. The disclosure of this U.S. patent is incorporated by reference herein.

WO2015/140782, entitled “Biopsy Method and Clinic for Imaging new Biopsy”, published 24 Sep. 2015, describes and claims a method of preparing tissue for biopsy comprising: restraining non-rigid tissue by applying pressure to the non-rigid tissue; imaging restrained non-rigid tissue to provide at least one image thereof; releasing the non-rigid tissue; re-restraining the non-rigid tissue by applying pressure to the non-rigid tissue, after the imaging; and registering re-restrained tissue with the at least one image.

U.S. Pat. No. 9,332,947, entitled “X-ray mammography and/or breast tomosynthesis using a compression paddle with an inflatable jacket with dual bottom layer joined at a seam enhancing imaging and improving patient comfort” issued on 10 May 2016. It describes and claims an x-ray breast imaging system comprising: a data acquisition unit comprising an x-ray source configured to selectively emit an imaging x-ray beam, an image receptor configured to receive said beam and produce a plurality of x-ray imaging information in response thereto, and a breast immobilizer between the source and the receptor; said breast immobilizer comprising a breast platform configured to support a patient's breast for imaging with said beam and a compression paddle supported for movement toward the breast platform to compress the breast and away from the breast platform to release the breast; said compression paddle having a front wall configured to be adjacent the patient's chest wall when the patient's breast is supported for said imaging, side walls extending transversely to the front wall, and an underside facing the breast platform; a paddle jacket removably secured to the compression paddle, said jacket having a first flexible sheet and a second flexible sheet joined at a seam, wherein the first flexible sheet and the second flexible sheet extend along the underside of the paddle when secured to the paddle and form an inflatable chamber, and a fluid conduit that is in fluid flow communication with said chamber; a fluid control unit releasably coupled with said conduit, said fluid control unit configured to selectively supply fluid to said chamber through said conduit and thereby selectively inflate and deflate the chamber, said fluid control unit further configured to maintain a substantially uniform internal pressure throughout an imaging procedure; an image processor coupled with said image receptor and configured to receive said imaging information therefrom and produce x-ray images; and a workstation and system control unit coupled with said data acquisition unit, said fluid control unit and said image processor, and configured to control operations thereof in response to operator inputs. The disclosure of this U.S. patent is incorporated by reference herein.

Other documents in the area of support for breasts during breast biopsy procedures include, but are not limited to, U.S. Pat. No. 3,963,933 entitled “Mammography Fixture”, issued 15 Jun. 1976; U.S. Pat. No. 5,660,185, entitled “Image-guided biopsy apparatus with enhanced imaging and methods”, issued 26 Aug. 1997; U.S. Pat. No. 5,702,405, entitled “Stereotactic auxiliary attachment for a tomography apparatus for Tomogram Guided Implementation of a Biopsy” issued 30 Dec. 1997; U.S. Pat. No. 6,589,254 entitled “Breast Bracket”, issued 8 Jul. 2003; U.S. Pat. No. 7,740,593 entitled “Guide Block for Biopsy or Surgical Devices”, issued 22 Jun. 2010; U.S. Pat. No. 8,532,745 entitled “Breast Biopsy and Needle Localization Using Tomosynthesis Systems”, issued 10 Sep. 2013; and U.S. Pat. No. 8,292,824, entitled “Biopsy Device”, issued 23 Oct. 2012. The disclosure of each of the above-cited U.S. patents is incorporated by reference herein.

Other non-US references in this field include DE29908202U1, “Medical Compression Device” 2 Sep. 1999. The German language abstract for this patent application is copied here and is followed by two different machine translations of the abstract:

German Language Abstract: Medizinische Kompressionsvorrichtung, umfassend zwei mit einem variablen Abstand angeordnete Kompressionsplatten (1) zum Komprimieren eines Untersuchungsobjekts (3), das über eine Zugangsöffnung (14) in einen sich zwischen den Kompressionsplatten befindenden Untersuchungsraum (2) einbringbar ist, dadurch gekennzeichnet, daß die Kompressionsplatten (1) auf ihrer dem Untersuchungsraum zugewandten Seite jeweils mit einer elastischen Folie (4A) verbunden sind, daß jede Folie (4A) mit der damit verbundenen Seite der Kompressionsplatte (1) einen Innenraum (5, 5Aa) begrenzt, daß die Innenräume (5, 5A) mit mindestens einem Flüssigkeitsbehälter (7, 7A) verbunden sind, der mit einer Flüssigkeitspumpvorrichtung (9) verbunden ist zum Füllen und Entleeren der Innenräume oder randseitig mit einer in Richtung des variablen Abstandes der Kompressionsplatten größenvariablen Wandung (11A) verbunden sind und die Wände relativ starr sein sollen, daß die Zugangsöffnung (14) von einer elastischen Folie (4) abgedeckt ist, daß die Kompressionsplatten (1), die Wandung (11A) und die Folie (4) einen flüssigkeitsdichten Innenraum (5) begrenzen, daß die Folie (4) mit einem stangenförmigen Element (26) verbunden ist und zum Verformen der Folie (4) von der Zugangsöffnung (14) in den Innenraum (5) hinein, daß in die Folie (4) nach auBen eine durch den Innenraum (5) geführte Leitung mündet und daß der Innenraum (5) mit einem Flüssigkeitsbehälter (7) verbunden ist oder an seitlichen und unteren Rändern (29, 30) mit einer in Richtung des variablen Abstandes größenvariablen Wandung (11) verbunden sind und die Wände relativ starr sein sollen, daß mit Hilfe von Dichtmitteln (4B, 35) die Kompressionsplatten zusammen mit der Wandung und dem durch die Zugangsöffnung und das eingebrachte Untersuchungsobjekt einen bis auf die Zugangsöffnung flüssigkeitsdichten Untersuchungsraum (2) bilden und daß ein Flüssigkeitsbehälter (7) über Flüssigkeitsleitungen (6C) mit dem flüssigkeitsdichten Untersuchungsraum verbunden ist. First Machine Translation of German Language Abstract into English: Medical compression device comprising two spaced with a variable distance between compression plates (1) for compressing an object to be examined (3), which can be introduced through an access opening (14) in a located between the compression plates examination space (2), characterized in that the compression plates (1): “Device 1 (FIG. 1/1-1/9)” Are first connected on its side facing the examination space-facing side in each case with an elastic foil (4 A), in that each slide (4 A) with the associated side of the compression plate (1) an interior space (5, 5 Aa) limited such that the interior (5, 5 A) with at least one liquid container (7, 7 A) are connected, which is connected with a fluid pumping device (9) for filling and emptying of the interiors or “Device 2 (FIG. 2/1-2/3)” Second edge having a size variable in the direction of the variable spacing of the compression plates wall (11 A) are connected and the walls should be relatively rigid in that the access opening (14) of an elastic foil (4) is covered, that the compression plates (1), the wall (11 A) and the film (4) has a liquid-tight interior space (5) limited in that the film (4) with a rod-shaped element (26) and for deforming the foil (4) from the access opening (14) into the interior (5) in that in the film (4) to the outside through the interior of a (5) line opens out and in that the interior (5) with a liquid container (7) is connected or Device 3 (FIG. 3/1-3/9) at edges of lateral and lower (29, 30) are connected with a size variable in the direction of the variable spacing wall (11) and the walls should be relatively rigid, that with the aid of sealing means (4 B, 35), the compression plates together with the forming wall and through the access opening and the inserted object to be examined a liquid-tight except for the access opening of the examination room (2) and that a liquid container (7) via fluid lines (6 C) is connected to the liquid-tight examination room.

Second Machine Translation of German Language Abstract: Medical compression device comprising two spaced with a variable spacing compression plates (1) for compressing an object to be examined (3), which can be introduced through an access opening (14) in a located between the compression plates examination space (2), characterized in that the compression plates (1) are connected on its side facing the examination space-facing side in each case with an elastic film (4A), that each film (4A) with the associated side of the compression plate (1) an interior space (5, 5Aa) is limited such that the internal spaces (5, 5A) with at least one liquid container (7, 7A) are connected, which is connected to a liquid pump device (9) for filling and emptying of the interiors or the edge side with a size variable in the direction of the variable spacing of the compression plates wall (11A) are connected and the walls should be relatively rigid in that the access opening (14) of an elastic foil (4) is covered, that the compression plates (1), the wall (11A) and the film (4) define a liquid-tight interior space (5), in that the film (4) is connected to a rod-shaped element (26) and for deforming the foil (4) from the access opening (14) into the interior (5) into that in the sheet (4) outwards a through the interior (5) led line opens and that the inner space (5) is connected to a liquid container (7) or on lateral edges and bottom (29, 30) are connected to a size variable in the direction of the variable spacing wall (11) and the walls relatively rigid should be that form with the aid of sealing means (4B, 35), the compression plates together with the wall and the through the access opening and the inserted object to be examined a liquid-tight except for the access opening examination room (2) and that a liquid container (7) via fluid lines (6C) is connected to the liquid-tight examination room

Known breast biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; and U.S. Pat. No. 8,118,755, entitled “Biopsy Sample Storage,” issued Feb. 21, 2012. The disclosure of each of the above-cited U.S. patents is incorporated by reference herein.

More known biopsy devices and biopsy system components are disclosed in U.S. Pat. Pub. No. 2006/0074345, entitled “Biopsy Apparatus and Method,” published Apr. 6, 2006; U.S. Pat. Pub. No. 2008/0146962, entitled “Biopsy System with Vacuum Control Module,” published Jun. 19, 2008; U.S. Pat. Pub. No. 2008/0214955, entitled “Presentation of Biopsy Sample by Biopsy Device,” published Sep. 4, 2008; U.S. Pat. Pub. No. 2008/0221480, entitled “Biopsy Sample Storage,” published Sep. 11, 2008, issued as U.S. Pat. No. 8,118,755 on Feb. 21, 2012; U.S. Pat. Pub. No. 2009/0131821, entitled “Graphical User Interface For Biopsy System Control Module,” published May 21, 2009; U.S. Pat. Pub. No. 2009/0131820, entitled “Icon-Based User Interface on Biopsy System Control Module,” published May 21, 2009, issued as U.S. Pat. No. 8,454,531 on Jun. 4, 2013; U.S. Pat. Pub. No. 2010/0113973, entitled “Biopsy Device with Rotatable Tissue Sample Holder,” published May 6, 2010, issued as U.S. Pat. No. 8,241,226 on Aug. 14, 2012; U.S. Pat. Pub. No. 2010/0152610, entitled “Hand Actuated Tetherless Biopsy Device with Pistol Grip,” published Jun. 17, 2010; U.S. Pat. Pub. No. 2010/0160819, entitled “Biopsy Device with Central Thumbwheel,” published Jun. 24, 2010; U.S. Pat. Pub. No. 2010/0160824, entitled “Biopsy Device with Discrete Tissue Chambers,” published Jun. 24, 2010, issued as U.S. Pat. No. 8,702,623 on Apr. 22, 2014; U.S. Pat. Pub. No. 2010/0317997, entitled “Tetherless Biopsy Device with Reusable Portion,” published Dec. 16, 2010, issued as U.S. Pat. No. 8,206,316 on Jun. 26, 2012; U.S. Pat. Pub. No. 2012/0109007, entitled “Handheld Biopsy Device with Needle Firing,” published May 3, 2012; U.S. Non-Provisional patent application Ser. No. 13/086,567, entitled “Biopsy Device with Motorized Needle Firing,” filed Apr. 14, 2011, published as U.S. Pat. Pub. No. 2012/0265095 on Oct. 18, 2012; U.S. Non-Provisional patent application Ser. No. 13/150,950, entitled “Needle Assembly and Blade Assembly for Biopsy Device,” filed Jun. 1, 2011, published as U.S. Pat. Pub. No. 2012/0310110 on Dec. 6, 2012; U.S. Non-Provisional patent application Ser. No. 13/205,189, entitled “Access Chamber and Markers for Biopsy Device,” filed Aug. 8, 2011, published as U.S. Pat. Pub. No. 2013/0041256 on Feb. 14, 2013; U.S. Non-Provisional patent application Ser. No. 13/218,656, entitled “Biopsy Device Tissue Sample Holder with Bulk Chamber and Pathology Chamber,” filed Aug. 26, 2011, published as U.S. Pat. Pub. No. 2013/0053724 on Feb. 28, 2013; U.S. Provisional Patent App. No. 61/566,793, entitled “Biopsy Device With Slide-In Probe,” filed Dec. 5, 2011; and U.S. Non-Provisional patent application Ser. No. 13/483,235, entitled “Control for Biopsy Device,” filed May 30, 2012, published as U.S. Pat. Pub. No. 2013/0324882 on Dec. 5, 2013. The disclosure of each of the above-cited U.S. patent application Publications, U.S. Non-Provisional patent applications, and U.S. Provisional Patent Applications is incorporated by reference herein.

The prior art appears to be focused on unmeasured, “ad-hoc” solutions to increasing breast compression from below the nipples. Applicant is unaware of any existing solution that provides a means for the clinician to set a measured amount of upward compressive force upon the breast during the biopsy. Applicant is aware that existing support systems do not allow for remote adjustment of the pressure to fine-tune breast compression at the insertion site. Currently to adjust pressure at the insertion site, the patient is typically moved out of position in order to for the clinician to adjust the support which can lengthen the time the procedure takes, cause patient discomfort and has the potential to disrupt the targeting alignment that has already been established.

While several systems and methods have been made and used for supporting a breast during a breast biopsy procedure, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of a biopsy system including a control module remotely coupled to a biopsy device, and including a localization fixture with a lateral grid plate used in conjunction with a rotatable cube to position an obturator or a probe of the biopsy device to a desired insertion depth as set by a ring stop;

FIG. 2 depicts a perspective view of a breast coil receiving the localization fixture of FIG. 1;

FIG. 3 depicts a perspective view of the biopsy device inserted through the rotatable cube within the cube plate of the localization fixture attached to the breast coil of FIG. 2;

FIG. 4 depicts a perspective view of a patient support device for use with the biopsy system of FIG. 1;

FIG. 5 depicts a front elevational view of an expandable member of the patient support device of FIG. 4, with the expandable member disposed within the localization fixture of FIG. 1 in a collapsed state;

FIG. 6 depicts another front elevational view of the expandable member of FIG. 5, with a patient's breast adjacent to the expandable member;

FIG. 7 depicts a still another front elevational view of the expandable member of FIG. 5, with the expandable member in a pressurized state;

FIG. 8 depicts a yet another front elevational view of the expandable member of FIG. 5, with the expandable member in the pressurized state and the breast clamped;

FIG. 9 depicts a perspective view of a grid plate of the localization fixture of FIG. 1 adjacent to a patient's breast; and

FIG. 10 depicts another perspective view of the grid plate of FIG. 9 and the expandable member of FIG. 5, with the expandable member in the fully pressurized state.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### SUMMARY OF THE INVENTION

The first aspect of the instant claimed invention is an apparatus for use with a biopsy localization fixture, the localization fixture comprising a grid plate and a medial plate, wherein the apparatus comprises: a support member, wherein the support member is configured to be positionable between the grid plate and the medial plate, wherein the support member is further configured to abut a patient's breast to apply an upward force to the breast thereby spreading the breast laterally across the grid plate; and an actuation device, wherein the actuation device is in communication with the support member.

The second aspect of the instant claimed invention is a bolstering device, wherein the bolstering device is usable in conjunction with a breast biopsy localization fixture, wherein the localization fixture comprise two opposing plates configured for compression of a patient's breast therebetween, wherein the bolstering device is positionable between the two opposing plates, wherein the bolstering device comprises: a flat, or curved, top surface; a flat bottom surface; and a plurality of ribs disposed between the top surface and the bottom surface, wherein the plurality of ribs are configured to expand substantially unidirectionally to push the top surface upwardly thereby applying a force to a patient's breast, wherein the force applied by the top surface is oriented along a force axis, wherein the force axis is obliquely oriented relative to a compression axis defined the two opposing plates of the localization fixture.

The third aspect of the instant claimed invention is an apparatus for use with a biopsy localization fixture, the localization fixture comprising a grid plate and a medial plate, wherein the apparatus comprises: an expandable member, wherein the expandable member is configured to be disposed between the grid plate and the medial plate, wherein the expandable member is further configured to expand relative to the grid plate and the medial plate in a direction perpendicular to a localization axis; a tube; and a pressure source, wherein the pressure source is in communication with the expandable member via the tube, wherein the pressure source is configured to selectively supply fluid to the expandable member to selectively expand the expandable member in the direction perpendicular to the localization axis.

The fourth aspect of the instant claimed invention is a method for positioning a breast of a patient in preparation for a biopsy procedure, the method comprising the steps of: actuating a first assembly to bear against the breast in a first orthogonal direction and thereby deform the breast; and actuating a second assembly to bear against the breast in a second orthogonal direction that is perpendicular to the first orthogonal direction to thereby further deform the breast.

### DETAILED DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

The invention provides an adjustable means for supporting the breast from beneath the nipple during the prone MRI biopsy procedure. By increasing or decreasing the volume of air or fluid within the pillow, the clinician can apply a controlled amount of compression on the breast, thereby increasing or decreasing the compressed area of the breast.

Parts List

| Number | Description |
|---|---|
| 10 | Biopsy System |
| 12 | Control Module |
| 14 | Biopsy Device |
| 15 | Localization Assembly |
| 16 | Localization Fixture |
| 18 | Breast Coil |
| 20 | Cable Management Spool |
| 22 | Saddle |
| 24 | Electrical Cable |
| 26 | Mechanical Cable |
| 28 | Electrical Port |
| 30 | Mechanical Port |
| 32 | Holster Portion |
| 34 | Docking Cup |
| 36 | Mounting Bracket |
| 38 | Interface Lock Box |
| 40 | Tether |
| 42 | Lockout port |
| 44 | In-line Enclosure |
| 46 | Vacuum Line |
| 48 | Outlet Port |
| 50 | Canister |
| 52 | Tubing Kit |
| 54 | Second Vacuum Line |
| 56 | Inlet Port |
| 58 | Vacuum Line (2nd Vacuum |
| | Line) |
| 60 | Vacuum Line (2nd Vacuum |
| | Line) |
| 62 | Remote Keypad |
| 63 | Thumbwheel (Aft End) |
| 64 | Left Upper Guide |
| 66 | Right Upper Guide |
| 68 | Localization Framework |
| 70 | Left Upper Track |
| 72 | Right Upper Track |
| 74 | Underside |
| 76 | Breast Aperture |
| 78 | Patient Support Platform |
| 80 | Base |
| 82 | Centerline Pillars |
| 84 | Vertical Support Pillar |
| 86 | Vertical Support Pillar |
| 88 | Lateral Recess |
| 90 | Needle |
| 91 | Probe |
| 92 | Obturator |
| 94 | Cannula |
| 95 | Depth Stop Device (Z-Stop) |
| 96 | Grid Plate |
| 98 | Plate Bracket |
| 100 | Medial Plate |
| 102 | Plate Bracket |
| 104 | Guide Cube |
| — | — |
| 200 | Patient Support Device |
| 210 | Tube |
| 212 | Pressure Source |
| 220 | Expandable member |
| 222 | Flat or Curved Top Surface |
| 224 | Flat Bottom Surface |
| 226 | Annular Folds |

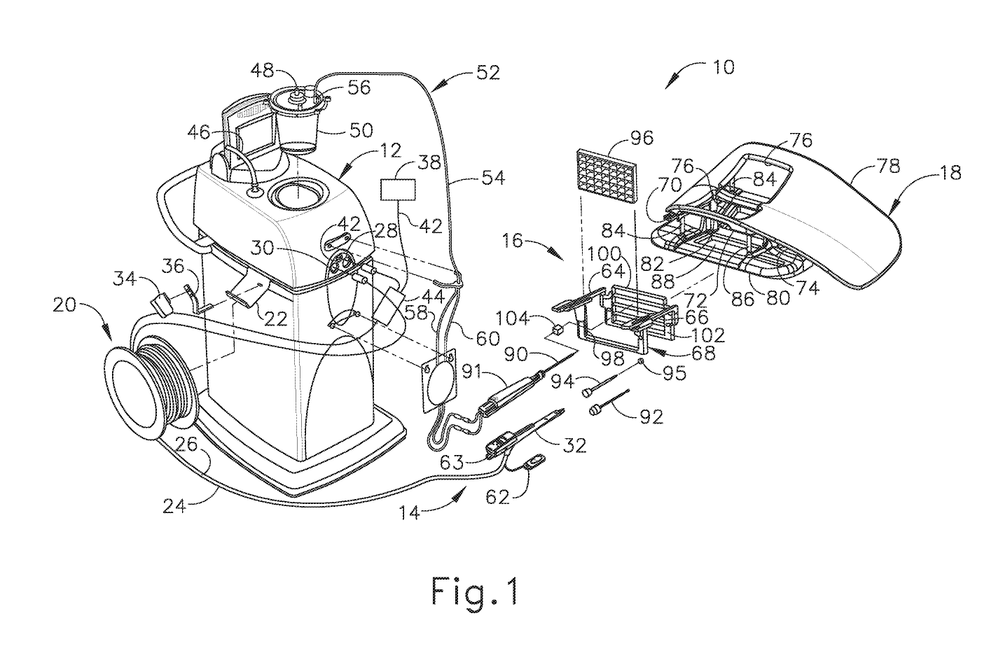

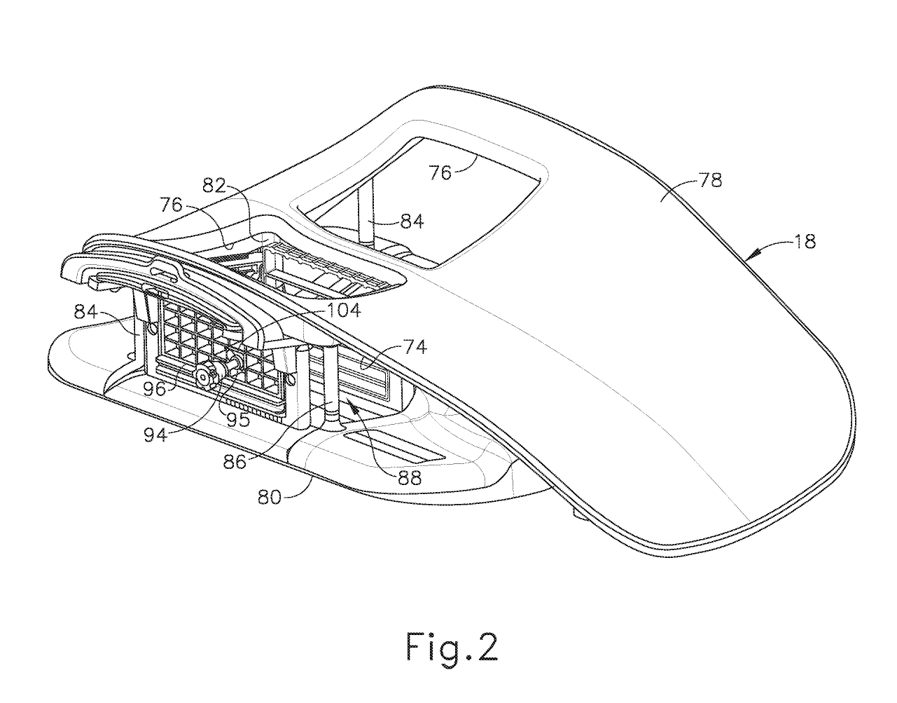

In FIGS. 1-3, MRI compatible biopsy system (10) has control module (12) that may be placed outside of a shielded room containing an MRI machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MRI machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MRI machine or other machine. In-line enclosure (44) may register tether (40), electrical cable (24) and mechanical cable (26) to their respective ports (42, 28, 30) on control module (12).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pat. No. 7,938,786, entitled “Vacuum Timing Algorithm for Biopsy Device,” issued May 10, 2011, the disclosure of which is incorporated by reference herein. As another merely illustrative example, control module (12) may instead be configured and operable in accordance with the teachings of U.S. Pat. No. 8,328,732, entitled “Control Module Interface for MRI Biopsy Device,” issued Dec. 11, 2012, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

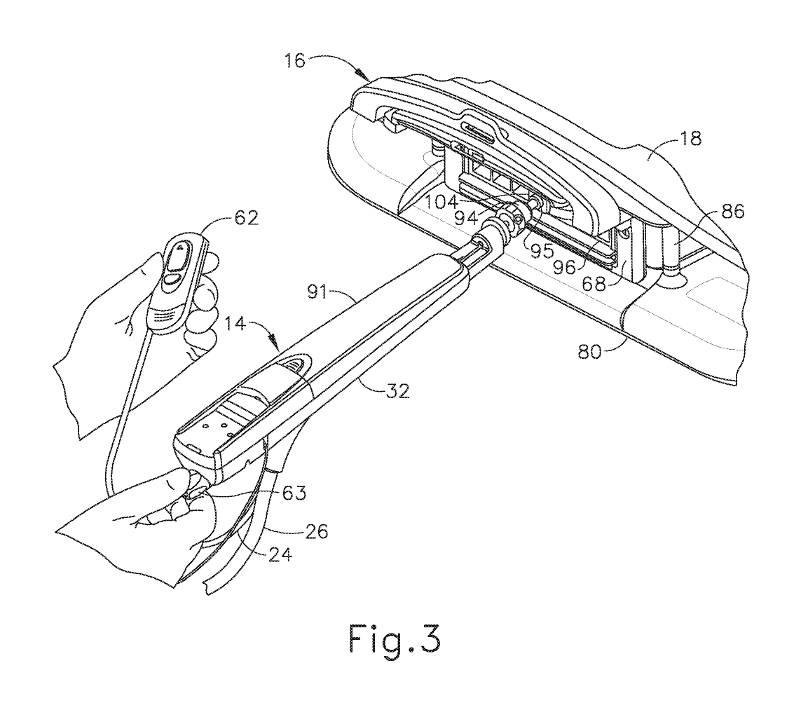

In the present example, cannula (94) and obturator (92) are associated with probe (91). In particular, obturator (92) is slid into cannula (94) and the combination is guided through guide cube (104) to the biopsy site within the breast tissue. As shown in FIG. 3, obturator (92) is then withdrawn from cannula (94), then needle (90) of probe (91) is inserted in cannula (94), and then biopsy device (14) is operated to acquire one or more tissue samples from the breast via needle (90).

Cannula (94) of the present example is proximally attached to a cylindrical hub and cannula (94) includes lumen (not shown) and lateral aperture (not shown) proximate to an open distal end. In some examples, the cylindrical hub has an exteriorly presented thumbwheel for rotating the lateral aperture of cannula (94). The cylindrical hub may also include an interior recess that encompasses duckbill seal, wiper seal, and seal retainer to provide a fluid seal when the lumen of cannula (94) is empty and for sealing to inserted obturator (92). Longitudinally spaced measurement indicia (not shown) along an outer surface of cannula (94) visually, and perhaps physically, provide a means to locate depth stop device (95) of FIG. 1.

Obturator (92) of the present example incorporates a number of components with corresponding features. For instance, in some examples obturator (92) includes a fluid lumen (not shown) that communicates between an imagable side notch and a proximal port. Additionally, in some example obturator (92) is longitudinally sized such that a piercing tip positioned on the distal end of obturator (92) extends out of the open distal end of cannula (94).

While obturator (92) is hollow in some examples, it should be understood that obturator (92) may alternatively have a substantially solid interior, such that obturator (92) does not define an interior lumen. In addition, obturator (92) may omit the side notch in some versions. Other suitable components, features, configurations, functionalities, operability, etc. for an obturator (92) will be apparent to those of ordinary skill in the art in view of the teachings herein. Likewise, cannula (94) may be varied in a number of ways. For instance, in some other versions, cannula (94) has a closed distal end. As another merely illustrative example, cannula (94) may have a closed piercing tip instead of obturator (92) having a piercing tip. In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop (95). Depth stop may be of any suitable configuration that is operable to prevent cannula (94) and obturator (92) (or needle (90)) from being inserted further than desired. For instance, depth stop (95) may be positioned on the exterior of cannula (94) (or needle (90)), and may be configured to restrict the extent to which cannula (94) is inserted into a guide cube. It should be understood that such restriction by depth stop (95) may further provide a limit on the depth to which the combination of cannula (94) and obturator (92) (or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stops (95) that may be used with biopsy system (10) are described in U.S. Pat. No. 8,568,333, entitled “Grid and Rotatable Cube Guide Localization Fixture for Biopsy Device,” issued Oct. 29, 2013, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with the lateral aperture of cannula (94) when needle (90) is inserted into lumen of cannula (94). Probe (91) of the present example further comprises a rotating and translating cutter (not shown), which is driven by components in holster (32), and which is operable to sever tissue protruding through the lateral aperture of cannula (94) and the lateral aperture of needle (90). Severed tissue samples may be retrieved from biopsy device (14) in any suitable fashion.

It should be understood that although biopsy system (10) is discussed above as utilizing disposable probe assembly (91), other suitable probe assemblies and biopsy device assemblies may be utilized. By way of example only, probe assembly (91) may be configured in accordance with at least some of the teachings of U.S. Pat. No. 8,206,316, entitled “Tetherless Biopsy Device with Reusable Portion,” issued Jun. 26, 2012, the disclosure of which is incorporated by reference herein; U.S. Pat. No. 8,277,394, entitled “Multi-Button Biopsy Device,” issued Oct. 2, 2012, the disclosure of which is incorporated by reference herein; and/or U.S. Pub. No. 2012/0065542, entitled “Biopsy Device Tissue Sample Holder with Removable Tray,” published Mar. 15, 2012, the disclosure of which is incorporated by reference herein. In other examples, probe assembly (91) may be configured in accordance with at least some of the teachings of U.S. Pat. No. 8,702,623, the disclosure of which is incorporated by reference herein; U.S. Pub. No. 2013/0144188, entitled “Biopsy Device with Slide-In Probe,” published Jun. 6, 2013, the disclosure of which is incorporated by reference herein; U.S. Pub. No. 2013/0324882, entitled “Control for Biopsy Device,” published Dec. 5, 2013, the disclosure of which is incorporated by reference herein; and/or U.S. Pub. No. 2014/0039343, entitled “Biopsy System,” published Feb. 6, 2014, the disclosure of which is incorporated by reference herein. Still other suitable forms of biopsy devices that may be used in conjunction with the various alternative components of system (10) as described herein will be apparent to those of ordinary skill in the art.

The following describes the Patient Support Device of the instant claimed invention. As described above, localization fixture (16) is insertable into lateral recess (88) of breast coil (18) to compress a patient's breast between grid plate (96) and medial plate (100) as the breast hangs pendulously through breast aperture (76). Such compressive force is applied primarily laterally to secure the breast for penetration by needle (90) and/or obturator (92) through grid plate (96). Because the compressive force applied by grid plate (96) and medial plate (100) is primarily lateral in direction (e.g., along the Z-axis of the Cartesian coordinate system described above), it should be understood that with some patients that anatomical structure of the breast may cause elongation of the breast downwardly in the anterior direction. In some instances, it may be desirable to prevent excessive anterior elongation of the breast. For instance, by preventing excessive anterior elongation, the breast may spread in the superior and inferior directions, thereby increasing the surface area of the breast that is accessible through grid plate (96). Therefore, in some examples it may be desirable to equip breast coil (18) with a patient support device that is configured to facilitate superior and inferior spreading of a patient's breasts

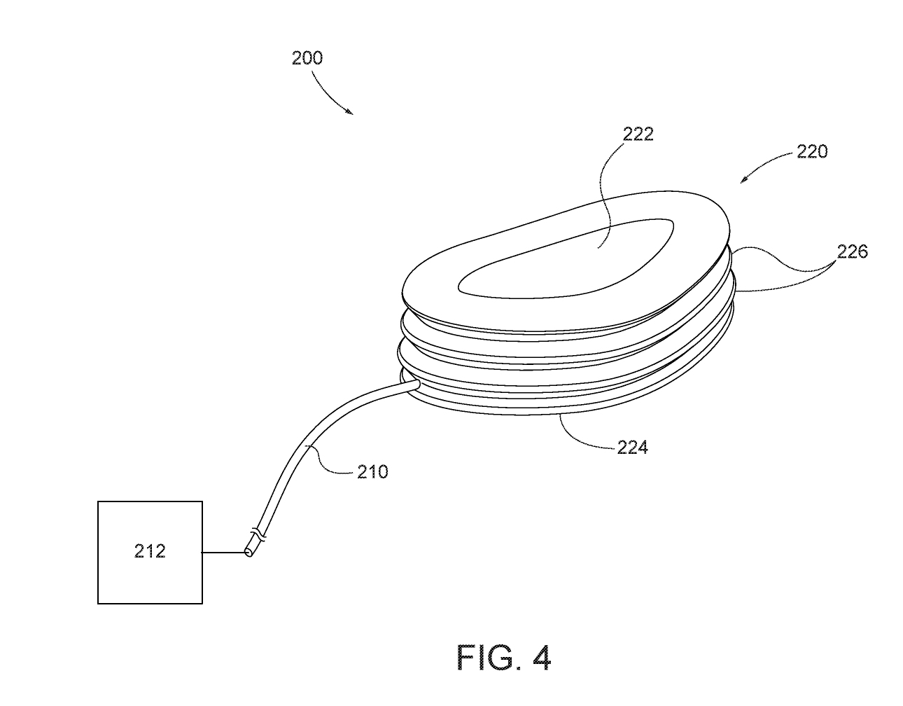

FIG. 4 shows an exemplary patient support device (200) that may be used in conjunction with breast coil (18) described above. As will be described in greater detail below, patient support device (200) is generally configured to fit within lateral recess (88) of breast coil (18) between grid plate (96) and medial plate (100). As will also be described in greater detail below, patient support device (200) is generally configured to expand vertically (or along the Y-axis) to provide anterior support to the breast, thereby facilitating superior and inferior spreading of the breast as the breast is compressed between grid plate (96) and medial plate (100).

Patient support device (200) comprises a tube (210), a pressure source (212), and an expandable member (220). Tube (210) of the present example comprises a hollow flexible tube that is configured to communicate fluid from pressure source (212) to expandable member (220). Pressure source (212) of the present example is configured to selectively deliver a pressurized fluid through tube (210) to expandable member (220).

By way of example only, pressure source (212) of the present example comprises a manually actuated syringe. It should be understood that such a syringe permits an operator to selectively fill (or evacuate) expandable member (220) with a desired amount of fluid. In other examples, the same functionality is provided by a manual or motor driven fluid pump or a foot-actuated pump. In still other examples, pressure source (212) comprises a pressurized reservoir pre-filled at a predetermined pressure level. In such an example, a pressure regulator is used to selectively fill expandable member (220) to a desired pressure level. In yet other examples, pressure source (212) is integrated into control module (12) such that tube (210) is in direct communication with control module (12). It should be understood that in such examples, pressure source (212) is selectively actuated using control module (12).

Expandable member (220) generally comprises an expandable or inflatable balloon-type structure shaped as an elongate cylinder. Expandable member (220) further comprises a flat or curved top surface (222) and a flat bottom surface (224). A plurality of annular folds (226) are disposed between top surface (222) and bottom surface (224) providing expandable member (220) with a bellows configuration. Annular folds (226) are configured to fold and expand relative to each other similar to a bellows to permit expansion of expandable member (220). However, each annular fold (226) is relatively fixed around its perimeter. Thus, expandable member (220) is generally free to expand vertically but remain relatively fixed horizontally.

Expandable member (220) is positioned relative to grid plate (96) and medial plate (100) such that expandable member (220) expands perpendicularly relative to a compression path defined by grid plate (96) and medial plate (100) (e.g., the path of medial plate (100) as it moves toward grid plate (96) to compression a patient's breast). As will be described in greater detail below, this positioning of expandable member (220) is configured to drive the breast upwardly along an axis that is perpendicular relative to the axis of compression provided by grid plate (96) and medial plate (100). Although expandable member (220) is described herein as being expandable along a path that is generally perpendicular to the axis of compression provided by grid plate (96) and medial plate (100), it should be understood that in other examples expandable member (220) can be configured to expand along a number of different axes. For instance, in some examples expandable member (220) expands along a path that is obliquely oriented relative to the axis of compression. In other examples, expandable member (220) expands along a path that is offset by 1 to 20° radially relative to the vertical path described above.

Although not shown, it should be understood that expandable member (220) comprises a generally hollow interior that is configured to be filled with fluid. Various suitable fluids are usable with expandable member (220). For instance, in the present example expandable member (220) is filled with atmospheric air. However, in other examples expandable member (220) is filled with an inert gas such as carbon dioxide or argon. In still other examples, expandable member (220) is filled with a liquid such as saline. Of course, other suitable fluids for use with expandable member (220) will be apparent to those of ordinary skill in the art in view of the teachings herein.

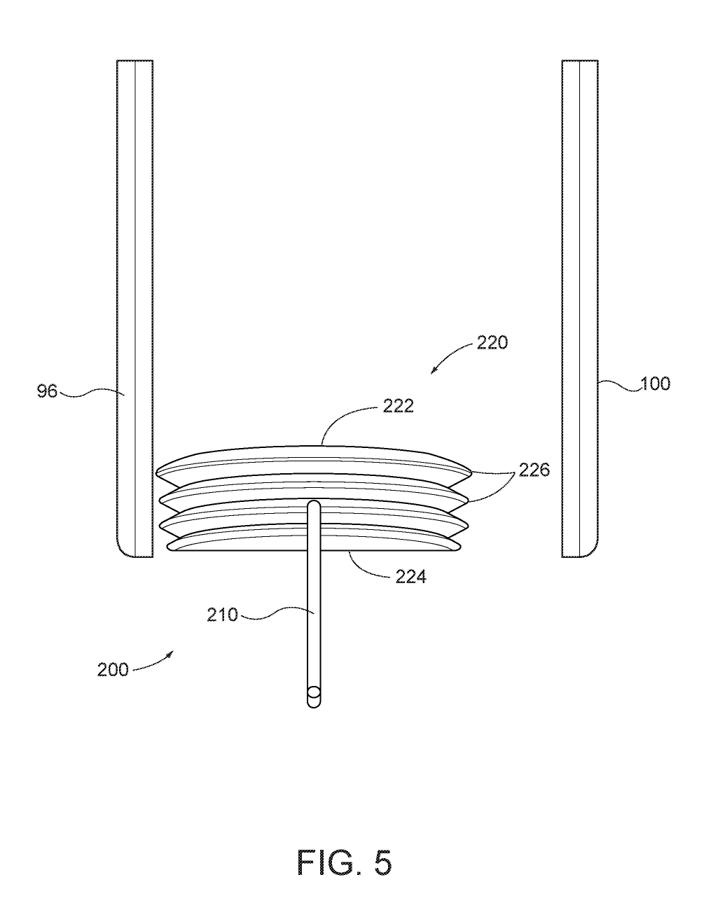

FIGS. 5-10 show an exemplary use of expandable member (220). As can be seen in FIG. 5, expandable member (220) of patient support device (200) is initially disposed between grid plate (96) and medial plate (100) of localization fixture (16). Furthermore, expandable member (220) is initially in a collapsed or deflated state to permit insertion of a patient's breast. It should be understood that although expandable member (220) is shown in FIG. 5 as being partially collapsed, in other examples expandable member (220) may be completely collapsed such that each annular fold (226) is fully folded into each adjacent annular fold (226). In such a state, expandable member (220) may occupy a minimum amount of vertical space corresponding to essentially the material thickness of expandable member (220).

While not shown, it should be understood that pressure source (212) may be positioned remotely from localization fixture (16) and/or breast coil (18) to enhance usability of localization fixture (16) and/or breast coil (18). As described above, in some examples pressure source (212) comprises a motor driven pump or other device that may include metals. In such examples, and where MRI equipment is used, pressure source (212) may be positioned a relatively large distance from breast coil (18) such that pressure source (212) does not interfere with sensitive imaging equipment. Of course, where such positioning is desired, tube (210) may be lengthened accordingly. Moreover, pressure source (212) and other components of patient support device (200) may be made entirely of non-ferrous material and/or otherwise MRI compatible material such that patient support device (200) does not present any risk of interference with MRI equipment.

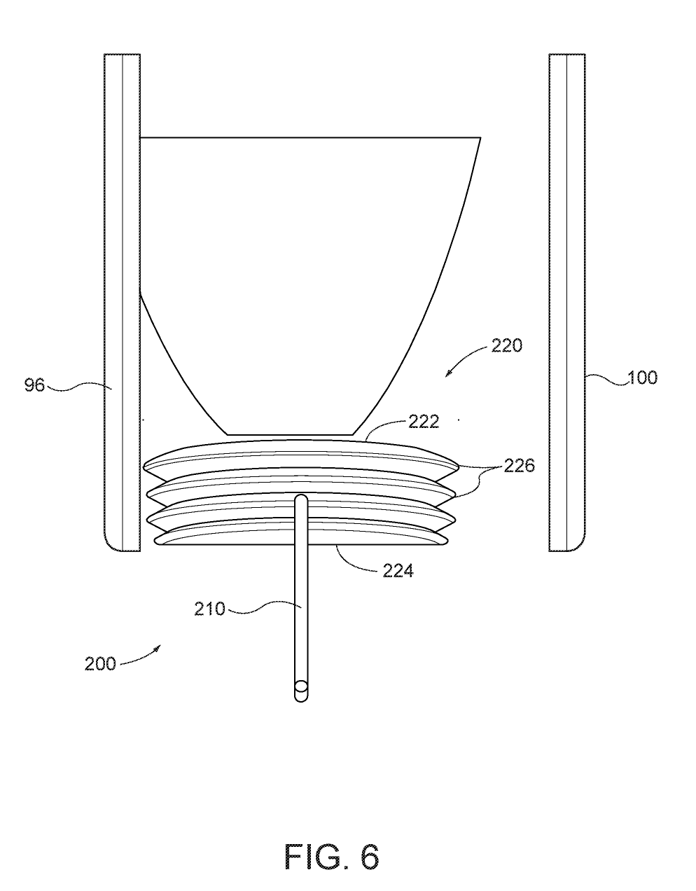

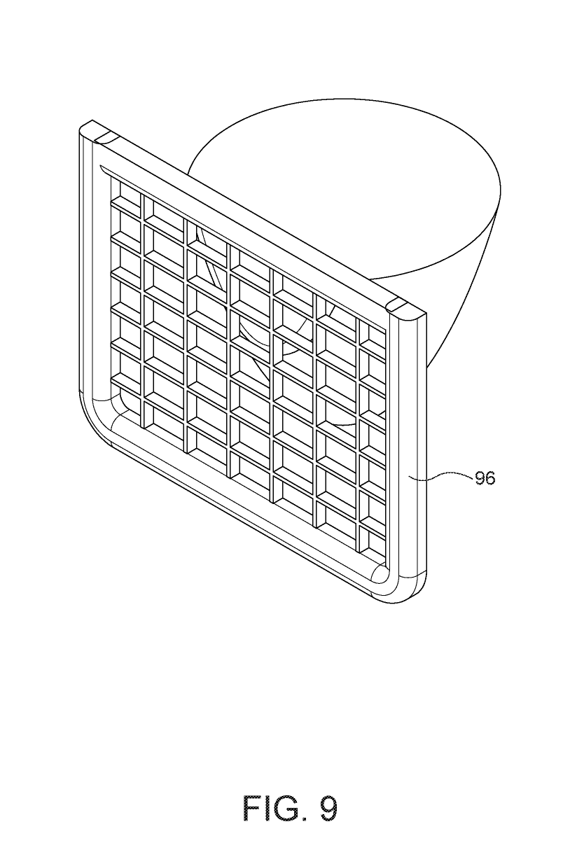

Once expandable member (220) has been positioned as desired between grid plate (96) and medial plate (100), a patient's breast may be inserted adjacent to expandable member (220) as shown in FIG. 6. As can be seen, with expandable member (220) in the collapsed state, a patient's breast is generally elongated anterior direction. As can be seen in corresponding FIG. 9, when a patient's breast is positioned in this way, a relatively small surface area of the breast abuts grid plate (96). Although a breast biopsy procedure may be performed in this state, it should be understood that in some instances additional surface area of the breast abutting grid plate (96) may be desirable.

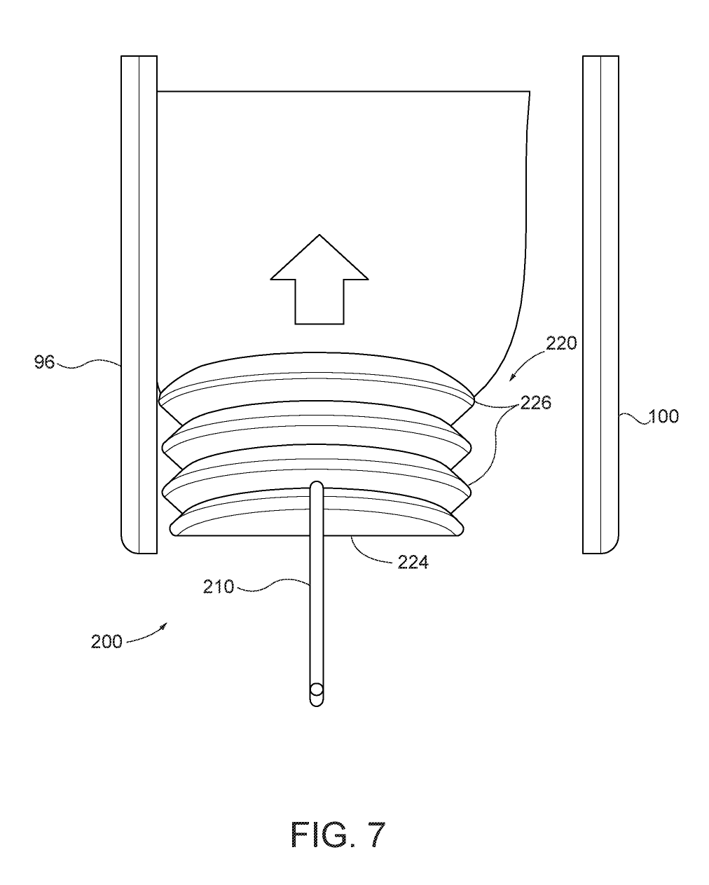

To increase the surface area of a patient's breast abutting grid plate, an operator may initiate actuation of pressure source (212). As pressure source (212) is actuated, expandable member (220) expands primarily in the vertical direction as shown in FIG. 7. As shown in FIG. 7, the vertical direction in the present example is oriented generally perpendicular to the normal axis of grid plate (96) and medial plate (100). In other words, expandable member (220) expands primarily in a direction that is perpendicular relative to the axis of compression defined by grid plate (96) and medial plate (100). This vertical expansion of expandable member (220) displaces at least a portion of a patient's breast upwardly or in the posterior direction. The particular amount of vertical expansion may be fine tuned by an operator using pressure source (212) to establish a desired amount of pressure within expandable member (220). Although expandable member (220) is described herein as being pressurized after insertion of a patient's breast, it should be understood that in some examples expandable member (220) is pre-pressurized prior to insertion of a patient's breast. The pressure may then be fine tuned or adjusted after insertion of a patient's breast using pressure source (212).

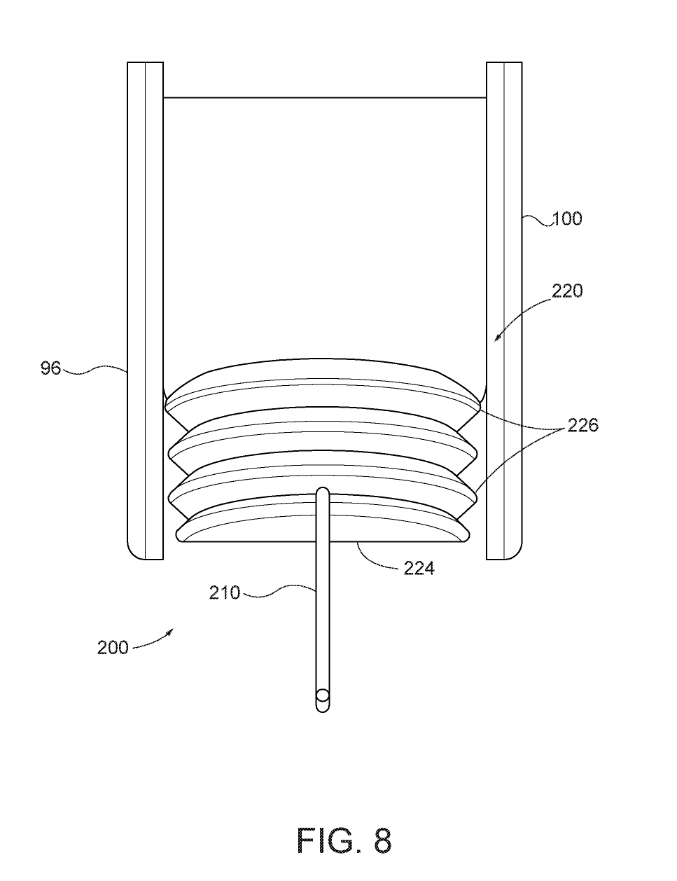

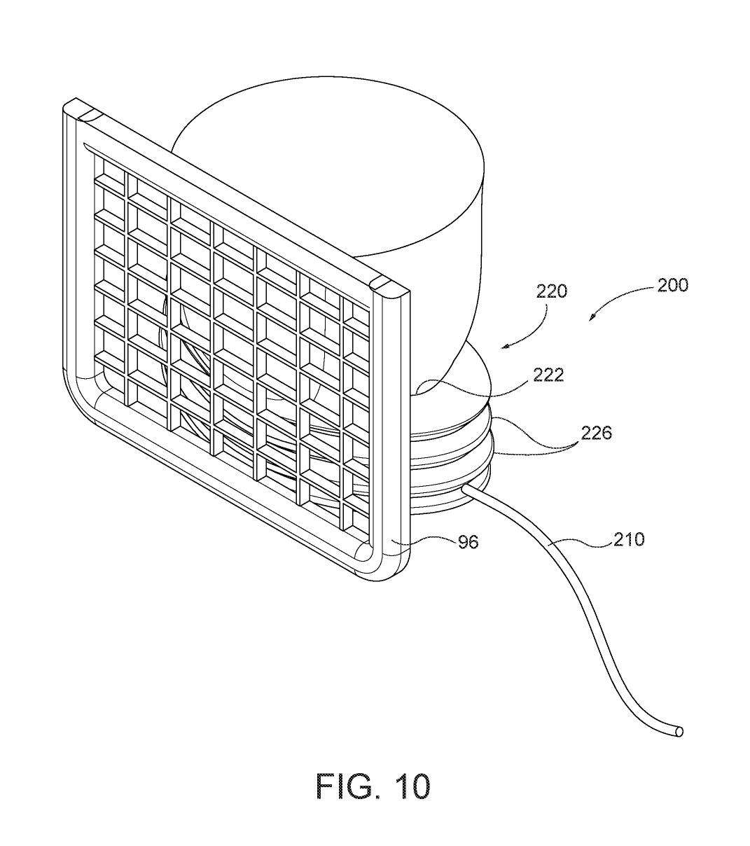

Once expandable member (220) is pressurized to a desired level, grid plate (96) and medial plate (100) are adjusted to compress a patient's breast between them. As shown in FIGS. 8 and 10, expandable member (220) acts to support the anterior portion of the breast to prevent expansion in the anterior direction. As is best seen in FIG. 10, this anterior support promotes medial and lateral spreading of the breast between grid plate (96) and medial plate (100) to maximize the surface area of the breast abutting grid plate (96). Accordingly, when grid plate (96) and medial plate (100) are used in conjunction with patient support device (200), an operator may have access to a wider area of the breast to perform a biopsy procedure.

Although patient support device (200) is described herein as being usable primarily in the context of MRI breast biopsy procedures, it should be understood that no such limitation is intended. For instance, patient support device (200) may be readily usable in other breast biopsy procedures where a patient's breast is immobilized such as stereotactic breast biopsy procedures. Of course, other procedures where patient support device (200) may be used will be apparent to those of ordinary skill in the art in view of the teachings herein.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I claim:

1. An apparatus for use with a biopsy localization fixture, the localization fixture comprising a grid plate and a medial plate, wherein the apparatus comprises:
(a) a support member, wherein the support member is configured to be positionable between the grid plate and the medial plate, wherein the support member is further configured to abut a patient's breast to apply an upward force to the breast thereby spreading the breast laterally across the grid plate; and
(b) an actuation device, wherein the actuation device is in communication with the support member.

2. The apparatus of claim 1, further comprising a tube, wherein the actuation device is in fluid communication with the support member via the tube.

3. The apparatus of claim 1, wherein the support member comprises a hollow interior, wherein the actuation device is configured to selectively supply a fluid to the support member.

4. The apparatus of claim 1, wherein the support member is configured to expand in a direction parallel to an anterior axis of the breast.

5. The apparatus of claim 1, wherein the support member comprises a plurality of ribs, wherein each rib of the plurality of ribs are configured to expand and retract relative the other ribs.

6. The apparatus of claim 3, wherein the fluid comprises atmospheric air.

7. The apparatus of claim 3, wherein the fluid comprises a liquid.

8. The apparatus of claim 1, wherein the actuation device is configured to selectively provide a plurality of predetermined pressure levels to the support member.

9. The apparatus of claim 8, wherein each pressure level of the plurality of pressure levels corresponds to a predetermined amount of upward force applied to a patient's breast.

10. The apparatus of claim 1, wherein the actuation device comprises a syringe.

11. The apparatus of claim 1, wherein the actuation device comprises a mechanical pump.

12. The apparatus of claim 11, wherein the mechanical pump is MRI compatible.

13. The apparatus of claim 1, wherein the actuation device comprises a pressure reservoir and a pressure regulator.

14. The apparatus of claim 1, wherein apparatus is configured for use in conjunction with an MRI imaging modality.

15. The apparatus of claim 1, wherein the apparatus is configured for use in conjunction with a stereotactic imaging modality.

16. The apparatus of claim 1, wherein the actuation device is configured to be positioned remotely from the support member.

17. The apparatus of claim 1, wherein the support member comprises a plurality of annular folds, wherein each annular fold of the plurality of annular folds are configured to expand and retract relative to the other annular folds.

18. A bolstering device, wherein the bolstering device is usable in conjunction with a breast biopsy localization fixture, wherein the localization fixture comprise two opposing plates configured for compression of a patient's breast therebetween, wherein the bolstering device is positionable between the two opposing plates, wherein the bolstering device comprises:
(a) a flat or curved top surface;
(b) a flat bottom surface; and
(c) a plurality of ribs disposed between the top surface and the bottom surface, wherein the plurality of ribs are configured to expand substantially unidirectionally to push the top surface upwardly thereby applying a force to a patient's breast, wherein the force applied by the top surface is oriented along a force axis, wherein the force axis is obliquely oriented relative to a compression axis defined the two opposing plates of the localization fixture.

19. The bolstering device of claim 18, wherein the top surface, the bottom surface, and the plurality of ribs define a hollow interior.

20. The bolstering device of claim 19, wherein the hollow interior is configured to be pressurized to a predetermined pressure to expand the plurality of ribs.

21. An apparatus for use with a biopsy localization fixture, the localization fixture comprising a grid plate and a medial plate, wherein the apparatus comprises:
(a) an expandable member, wherein the expandable member is configured to be disposed between the grid plate and the medial plate, wherein the expandable member is further configured to expand relative to the grid plate and the medial plate in a direction perpendicular to a localization axis;
(b) a tube; and
(c) a pressure source, wherein the pressure source is in communication with the expandable member via the tube, wherein the pressure source is configured to selectively supply fluid to the expandable member to selectively expand the expandable member in the direction perpendicular to the localization axis.

22. A method for positioning a breast of a patient in preparation for a biopsy procedure, the method comprising the steps of:
(1) actuating a first assembly to bear against the breast in a first orthogonal direction and thereby deform the breast; and
(2) actuating a second assembly to bear against the breast in a second orthogonal direction that is perpendicular to the first orthogonal direction to thereby further deform the breast.

23. The method of claim 22, wherein the first assembly comprises a pair of localization plates, wherein at least one of the localization plates is moved relative to the breast to actuate the first assembly.

24. The method of claim 22, wherein the second assembly comprises a support member having a plurality of annular folds, wherein each annular fold is movable relative to the other annular folds to actuate the second assembly.

25. The method of claim 24, wherein the step of actuating the second assembly further comprises inflating the support member with a fluid to move each annular fold relative to the other annular folds.

26. The method of claim 22, wherein the first orthogonal direction is perpendicular to the second orthogonal direction.
